SEQUENCE LISTING (2 sequences)

SEQ ID NO: 1 - protein, 7 aa, Artificial Sequence
  PG-901
XDPXRWK

SEQ ID NO: 2 - protein, 7 aa, Artificial Sequence
  PG-911
XDXXRWK

(19) United States
(12) Patent Application Publication
(10) Pub. No.: US 2012/0309677 A1
(43) Pub. Date: Dec. 6, 2012

(54) METHODS FOR REGULATING BLOOD GLUCOSE LEVELS

(75) Inventors: Michael Alexander Cowley, Balwyn (AU); Pablo Jose Enriori, Mount Eliza (AU); Russell Deputy Brown, Elwood (AU); Iain James Clarke, Malvern East (AU); Belinda Anne Henry, Murrumbeena (AU); Maria Cecilia Garcia-Rudaz, Mount Eliza (AU)

(73) Assignee: Monash University, Clayton (AU)

(21) Appl. No.: 13/575,165

(22) PCT Filed: Jan. 25, 2011
(86) PCT No.: PCT/AU2011/000075

§ 371 Date: Jul. 25, 2012

(30) Foreign Application Priority Data
Jan. 25, 2010 (AU) 2010900296

(51) Int. Cl. A61K 38/08; A61P 29/00; A61P 3/04; A61K 38/22; A61K 31/522; A61K 31/7076; G01N 33/566; A61P 3/10; A61K 31/7056

(52) U.S. Cl. 514/4.8; 514/6.8; 514/6.9; 514/43; 514/263.34; 514/47; 435/7.21

(57) ABSTRACT

The invention relates to methods and compositions for reducing blood glucose levels in hyperglycaemic subjects. The methods and compositions may therefore be suitable for treating a disease or condition associated with hyperglycaemia such as, for example, obesity (particularly diet induced obesity (DIO)), weight gain, Type II diabetes mellitus, insulin sensitivity, impaired glucose tolerance and inflammation. In some embodiments, the methods comprise administering a melanocortin-5 receptor (MC5R) agonist to one or more skeletal muscle cells of the subject. Preferred MC5R agonists are those that specifically activate MC5R and/or enhance expression of MC5R, such as the melanocortin analogue, Ac-Nle-c[Asp-Pro-D-Nal(2′)-Arg-Trp-Lys]-NH₂.

### FIELD OF THE INVENTION

The invention relates to methods and compositions for reducing blood glucose levels in hyperglycaemic subjects.

### INCORPORATION BY REFERENCE

This patent application claims priority from:
Australian Provisional Patent Application No 2010900296 titled “Methods for regulating blood glucose levels” filed 25 Jan. 2010.
   The entire content of this application is hereby incorporated by reference.

The following patent specifications are referred to herein:
U.S. Pat. No. 5,777,100 titled “AICA riboside analogs”,
U.S. Pat. No. 5,082,829 titled “AICA riboside prodrugs”,
United States Patent Publication No 2006/0287356 titled “Thienopyridones as AMPK activators for the treatment of diabetes and obesity”,
International Patent Publication No WO 2008/016278 titled “Furan-2-carboxylic acid derivatives and process for the preparation thereof”,
International Patent Publication No WO 2006/033709 titled “Novel nucleoside derivatives”,
International Patent Publication No WO 98/37097 titled “MSH-receptor subtype selective cyclic peptides”,
International Patent Publication No WO 2009/105824 titled “Methods of modulating the activity of the MC5 receptor and treatment of conditions related to this receptor”, and
Grieco, P et al., Biochem Biophys Res Commun 292(4):1075-1080 (2002).
   The entire content of these specifications and document is also hereby incorporated by reference.

### BACKGROUND TO THE INVENTION

Diabetes mellitus is a chronic disease that has no cure. In people with diabetes, the pancreas produces insufficient or no insulin, the hormone which is responsible for the absorption of glucose into cells for energy needs. As a result, the level of glucose in the blood becomes abnormally high (“hyperglycaemia”).

There are two main types of diabetes mellitus. Type 1, which is the more severe form, usually first appears in people under the age of 35 and develops rapidly. The insulin-secreting cells in the pancreas are destroyed and insulin production ceases almost completely. Without the regular administration of insulin the sufferer lapses into a coma and dies.

The most prevalent type of diabetes, Type II diabetes, is usually of gradual onset and occurs mainly in people over 40. Patients with Type II diabetes have this condition due to impaired utilisation or production of insulin. Endothelial dysfunction in patients with Type II diabetes can predispose the patients to atherosclerosis and target organ damage.

The long term complications of diabetes are a decreased life expectancy, neuropathy, an increased rate of blindness, an increased rate of kidney disease, an increased rate of heart disease, and an increased rate of peripheral and central vascular disease in comparison to nondiabetics. Maintaining proper blood glucose levels is therefore important for diabetics (ie hyperglycaemic subjects) in order to prevent long term problems such as nerve damage, blindness and kidney disease.

In people with diabetes, there are two specific types of hyperglycaemia that occur:
Fasting hyperglycaemia, which is defined as a blood sugar concentration greater than 90-130 mg/dL after fasting for at least 8 hours; and
Post-prandial or post-meal hyperglycaemia, which is defined as a blood sugar concentration usually greater than 180 mg/dL.

Obese subjects are commonly hyperglycaemic as a result of insulin resistance.

A variety of drugs are available for the treatment of diabetes mellitus, although none of them are without side effects. Insulin, even though very potent, remains the drug of choice for Type I diabetics and also for Type H diabetics who do not obtain glycemic control with oral anti-diabetic drugs.

There is a need for improved and/or alternative methods for reducing blood glucose levels in hyperglycaemic subjects.

### SUMMARY OF THE INVENTION

The present invention results from a finding that infusion of α-melanocyte stimulating hormone (α-MSH) into skeletal muscle leads to a rapid decrease in blood glucose levels. It has also now been found that α-MSH causes an increase of cAMP and pAMPK levels in muscle and that this leads to increased glucose uptake (with a concomitant decrease in blood glucose levels).

The present invention provides a method of reducing blood glucose levels in a hyperglycaemic subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

In some embodiments, the method of reducing blood glucose levels in a hyperglycaemic subject comprises administering α-MSH to one or more skeletal muscle cells of the subject.

In some embodiments, the method of reducing blood glucose levels in a hyperglycaemic subject comprises administering an AMPK agonist which increases the activity and/or expression of AMPK in skeletal muscle cells of the subject. An example of a suitable AMPK agonist is 5-aminoimidazole-4-carboxamide 1-β-D-ribonucleoside (AICAR).

In some embodiments, the method of reducing blood glucose levels in a hyperglycaemic subject comprises administering an agent which increases the level of cAMP in skeletal muscle cells of the subject. Suitable agents of this kind are preferably selected from the group consisting of: 3-isobutyl-1-methylxanthine (IBMX), and 8-bromoadenosine 3′,5′-cyclic monophosphate (8-Br-cAMP).

In some embodiments, the level of cAMP and/or activity of AMPK is increased in the hyperglycaemic subject by administering a melanocortin-5 receptor (MC5R) agonist to one or more skeletal muscle cells of the subject.

In a second aspect, the present invention provides a method of treating a disease or condition associated with hyperglycaemia in a subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

In a third aspect, the present invention provides a method of treating hyperglycaemia in an obese or overweight subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

In a fourth aspect, the present invention provides a pharmaceutical composition comprising an agent which: (i) increases the level of cyclic adenosine monophosphate (cAMP); and/or (ii) increases the activity of and/or expression of 5′ AMP-activated protein kinase (AMPK) in skeletal muscle of the subject optionally in combination with a pharmaceutically-acceptable carrier, diluent or excipient, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

### BRIEF DESCRIPTION OF THE ACCOMPANYING FIGURES

FIG. 1A provides graphical results showing that glucose and insulin treatments stimulate α-MSH release from adult monkey pituitaries. Monkey pituitaries (n=22) were halved and then treated separately with artificial cerebrospinal fluid (aCSF) equilibrated with 95% O₂ and 5% CO₂ and incubated at 37° C. After a 1 hr equilibration period, the halves were incubated for 45 min in aCSF (basal period) before being challenged with glucose (Glu): 15 mM, insulin (Ins): 200 nM or Glu+Ins (15 mM+200 nM) treatments for 45 min;

FIG. 1B provides graphical results of a radioimmunoassay showing that in monkey pituitaries, α-MSH was detected in the intermediate lobe (IL) and anterior lobe (AL). The neural lobe (NL) also showed traces of α-MSH;

FIG. 1C provides graphical results showing that children with hypopiuitarism (HP), and after craniopharyngioma (CP) had lower circulating α-MSH levels than healthy children (H). ***P<0.001;

FIG. 2A provides graphical results showing that α-MSH levels increase at 15 min. and sustained during 30 min in response to oral glucose administration, in both healthy (normal body weight) (---) and obese (—) humans. α-MSH levels were determined in 12 children treated at the Department of Pediatrics, University of Bonn (6 obese subjects: BMI>97th percentile versus 6 normal weight subjects, BMI 25-75th percentile) before and at different time-points after oral glucose loading (1.75 g/kg, max. 75 g). *P<0.05 vs baseline value in the corresponding group by repeated-measures ANOVA;

FIG. 2B provides graphical results showing that obese monkeys had a higher α-MSH response (more intense 2nd peak) to a glucose challenge than controls. α-MSH levels were measured during an intravenous glucose tolerance test in control (, n=6) and obese (▴, n=5). monkeys. *P<0.05 vs baseline value in the respective group by repeated-measures ANOVA. AUC was significantly higher in obese than control monkeys;

FIG. 2C provides graphical results showing that control (---) and obese mice (DIO) (—) showed a similar increase of α-MSH levels in response to glucose administration, to that observed in children. Glucose was administered intra-peritoneally (ipGTT). *P<0.05 vs baseline value in the respective group by repeated-measures ANOVA;

FIG. 3A provides graphical results showing that α-MSH levels flattened in response to glucose administration in POMC-Kir6.2 mutant mice (—) but not in control littermates (---). **P<0.01 vs baseline value in the respective group by repeated-measures ANOVA. # P<0.01 vs control littermate at the respective point by single measured;

FIG. 3B provides graphical results showing that α-MSH levels do not change in response to insulin neither in POMC-Kir6.2 mutant mice (light bars) nor in control littermates (dark bars);

FIG. 4A provides graphical results showing that systemic α-MSH infusion (external jugular vein) increases post-prandial muscle temperature in sheep. Animals were treated with α-MSH 100 μg/h (▾) or saline (800 μl/h, );

FIG. 4B provides graphical results showing that direct α-MSH infusion into femoral artery also causes an increase in muscle temperature. Animals were treated with α-MSH 1 μg/h (▾) or saline (800 μl/h, );

FIG. 5A provides graphical results showing that systemic α-MSH infusion (external jugular vein) increases glucose disposal during ipGTT in control mice;

FIG. 5B provides graphical results showing that systemic α-MSH infusion (external jugular vein) does not increase glucose disposal during ipGTT in obese (DIO) mice;

FIG. 5C provides graphical results showing that icy AgRP (to block MC4R) does not prevent the response (ie increased glucose disposal) to α-MSH infusion;

FIG. 5D provides graphical results showing that an MC5R-specific agonist (ie a melanocortin analogue denoted PG-901), increases glucose uptake in muscle cells;

FIG. 6A provides graphical results showing that α-MSH increases glucose uptake in soleus muscles from control mice when incubated in the presence and absence of insulin;

FIG. 6B provides graphical results showing that α-MSH (at the amount tested) does not increase glucose uptake in soleus muscles from obese mice when incubated in the presence and absence of insulin;

FIG. 7 provides graphical results showing that MC5R mRNA expression was similar in control and obese mice in baseline conditions. MC5R mRNA expression was assessed in soleus muscles of both control and DIO mice by RT-PCR;

FIG. 8 provides results showing that α-MSH treatment in vitro causes a dose-dependent increase of p-AMPK muscle levels from lean but not obese mice;

FIG. 9A provides graphical results showing that α-MSH infusion causes an increase of cAMP levels in muscles from control mice but not from obese mice. White bars: saline infusion, dark bars: α-MSH infusion;

FIG. 9B provides results showing that p-AMPK protein expression is increased in muscles from control but not from obese mice after α-MSH infusion. p-AMPK and total AMPK were determined by Western blot. Results are expressed as a ratio P/Total AMPK. *P<0.05; and

FIG. 10 provides graphical results showing that an enhanced increase in glucose uptake by rat L6 cells can be achieved with the combination of α-MSH and insulin.

### DETAILED DESCRIPTION OF THE INVENTION

The present invention provides a method of reducing blood glucose levels in a hyperglycaemic subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

As used herein the term “hyperglycaemic subject” and similar terms refers to a human or animal subject having excess glucose in the blood plasma. In humans, hyperglycaemic subjects usually have a blood glucose level that is in excess of 10 mmol/l. Type II diabetes can arise from, and be exacerbated by, obesity.

In work leading to the present invention, the present applicant has found that infusion, of the native peptide α-melanocyte stimulating hormone (α-MSH) increases glucose uptake in skeletal muscle (thus reducing blood glucose levels). Without intending to be bound by theory, it is herein proposed that α-MSH acts on the melanocortin-5 receptor (MC5R) to stimulate cAMP and activate (phosphorylate) AMPK and that this increases glucose uptake by muscle. This may be in parallel, and in addition to, insulin stimulated glucose uptake. Indeed, it is herein proposed that both glucose and insulin in the blood stimulates α-MSH secretion post-prandially by the pituitary gland, which then activates AMPK leading to increased glucose uptake by muscle, thermogenesis and fatty acid oxidation.

α-MSH is the native agonist for the type 1, the type 3, the type 4 and the type 5 melanocortin (MC) receptors. MSH peptides acting through stimulation of the MC receptors are known to have a variety of functions including immunomodulation, anti-inflammation, body temperature regulation, pain perception, aldosterone synthesis, blood pressure regulation, heart rate, vascular tone, brain blood flow, nerve growth, placental development, and synthesis/release of a variety of hormones such as aldosterone, thyroxin, prolactin and follicle stimulating hormone (FSH). However, a number of actions of MSH peptides, especially α-MSH, are not fully established with respect to which receptors are involved.

It has been suggested that α-MSH may be used to regulate body weight and/or weight gain (United States Patent Application No 2006/0063708). However, any metabolic function of α-MSH in this regard appears to be mediated via MC4R (unpublished results). In contrast, it is herein proposed that α-MSH decreases blood glucose levels by activating the MC5R pathway.

AMPK is a key integrator of hormone and nutrient signals that regulate energy balance. It seems likely that AMPK mediates contraction-stimulated glucose uptake in skeletal muscle¹.

In work leading to the present invention, the present applicant has found that infusion of α-MSH into skeletal muscle stimulates AMPK activity in the muscle. The increase in AMPK activity in the muscle results in an increase in glucose uptake into the muscle, thereby lowering blood glucose levels. However, the present applicant has also found that α-MSH does not have this effect in obese (hyperglycaemic) mice (at least, at the low amounts tested) and that α-MSH increases glucose uptake in soleus muscles from control mice but not from obese mice when incubated in the presence and absence of insulin. That is, obese (hyperglycaemic) individuals appear to have tissues that are resistant to α-MSH. Consequently, treatment of obese (hyperglycaemic) individuals with α-MSH would need to utilise relatively higher doses to that tested in the mice (eg≧100 nM) or, otherwise, treatment with α-MSH would be unlikely to result in a therapeutic reduction in blood glucose levels in those subjects. However, treatment with an agent that “bypasses” the α-MSH resistance to increase AMPK activity, can be useful for nevertheless reducing blood glucose levels in such individuals.

Therefore, in some embodiments, the method of reducing blood glucose levels in a hyperglycaemic subject comprises administering an AMPK agonist which increases the activity and/or expression of AMPK in the skeletal muscle cells of the subject.

Many previous studies suggesting a role for AMPK in the regulation of muscle glucose uptake are based on experiments using 5-aminoimidazole-4-carboxamide 1-β-D-ribonucleoside (AICAR), a compound that is converted to 5-aminoimidazole-4-carboxamide ribonucleotide (ZMP) within muscle². ZMP can then mimic the effect of AMP to increase AMPK activity³. Therefore, AICAR may be used to increase the level of AMPK in a subject and, accordingly, in some embodiments of the present invention, the AMPK agonist is AICAR.

The AMPK agonist can be any AMPK agonist, derivative, salt or ester thereof known to the person skilled in the art or yet to be developed. AMPK agonists other than AICAR are known in the art. For example, analogs of AICAR are disclosed in U.S. Pat. No. 5,777,100, and prodrugs or precursors of AICAR are disclosed in U.S. Pat. No. 5,082,829. Other activators of AMPK are disclosed in United States Patent Publication No 2006/0287356. Other AMPK agonists include leptin, adiponectin, metformin, DRL-16536 (Perlecan Pharma Pvt Ltd, Hyderabad, India), BG800 compounds (Betagenon AB, Stockholm, Sweden), furan-2-carboxylic acid derivative (International Patent Publication No WO 2008/016278), A-769662 (Abbott Laboratories Inc, Abbott Park, Ill., United States of America), AMPK agonists under development by Metabasis Therapeutics Inc. (La Jolla, Calif., United States of America) and described in International Patent Publication No WO 2006/033709, and the MT-39 series of compounds (Mercury Therapeutics, Inc., Woburn, Mass., United States of America).

An example of an agonist that specifically enhances expression of AMPK is a gene therapy agent comprising a polynucleotide molecule which comprises a polynucleotide sequence encoding AMPK.

In further work leading to the present invention, the present applicant has found that infusion of α-MSH into skeletal muscle also increases cAMP levels in the muscle of control subjects but does not have this effect in obese (hyperglycaemic) subjects (at least, at the low amounts tested). Consequently, another approach for bypassing the α-MSH resistance (to achieve a therapeutic reduction in blood glucose levels) in such individuals is to treat them with an agent which increases the level of cyclic adenosine monophosphate (cAMP).

Therefore, in some embodiments, the method of reducing blood glucose levels in a hyperglycaemic subject comprises administering an agent which increases the level of cyclic adenosine monophosphate (cAMP) in one or more skeletal muscle cells of the subject.

Adenylyl cyclase is an enzyme that catalyses the conversion of ATP to cAMP. Therefore, the level of cAMP may be increased by activating adenylyl cyclase. Consequently, in some embodiments, the level of cAMP is increased in the hyperglycaemic subject by administering an activator of adenylyl cyclase to the subject. Activators of adenylyl cyclase include forskolin. Other activators of adenylyl cyclase known in the art and/or under development could also be used. The activator of adenylyl cyclase may also be any derivative, salt or ester of activators of a known activator of adenylyl cyclase.

cAMP decomposition into AMP is catalysed by phosphodiesterase. Therefore, the level of cAMP may also be increased by inhibiting the activity of phosphodiesterase. In some embodiments, the level of cAMP is increased in the hyperglycaemic subject by administering a phosphodiesterase inhibitor to the subject. Phosphodiesterase inhibitors include methylated xanthanes such as 3-isobutyl-1-methylxanthine (IBMX).

Further, in some embodiments, the level of cAMP is increased in the hyperglycaemic subject by administering a protein kinase A (PKA) agonist to one or more skeletal muscle cells of the subject. PKA agonists include 8-bromoadenosine 3′,5′-cyclic monophosphate (8-Br-cAMP).

In work leading to the present invention, the present applicant has also found that melanocortin-5 receptor (MC5R) mRNA expression in muscle was similar in control and obese mice in baseline conditions. It is herein proposed, therefore, that α-MSH acts via MC5R in increasing the uptake of glucose into muscle. Therefore, the failure of α-MSH to result in uptake of glucose into the muscle of hyperglycaemic subjects may be due to the failure of α-MSH to stimulate signal transduction from MC5R. Consequently, any agent that is able to activate the MC5R pathway could result in uptake of glucose into muscle in vivo.

MC5R genes have been found to be expressed primarily in the hypothalamus, mid-brain and brainstem and in a wide distribution of peripheral tissues. Given the complexity of possible sites of expression of the MC3, MC4 and MC5 receptors, it has not been possible to unambiguously identify any simple correlation between these receptors and the reported biological activities of their ligands. Nevertheless, it is proposed that ligands of MC5R may activate the pathway and result in uptake of glucose into muscle. α-MSH is one such ligand and the work described hereinafter shows that administration of α-MSH results in glucose uptake into muscle.

Therefore, in some embodiments, the level of cAMP and/or AMPK is increased in the hyperglycaemic subject by administering a melanocortin-5 receptor (MC5R) agonist to one or more skeletal muscle cells of the subject.

Preferably, the MC5R agonist has little or no activity on other MC receptor types, particularly MC3R and MC4R. More preferably, the MC5R agonist specifically activates MC5R and/or enhances expression of MC5R.

Examples of agonists that specifically activate MC5R are MC5R selective melanotropin analogues such as that denoted PG-901, a highly potent and selective melanotropin analogue (EC₅₀=0.072 nm for hMC5R), and PG-911, which is another melanotropin analogue (EC₅₀+0.031 nm for hMC5R); these melanotropin analogues are described in Grieco, P et al., Biochem Biophys Res Commun 292(4):1075-1080 (2002)⁴, which is hereby incorporated by reference. They are analogues of the melanocortin fragment comprising amino acids 4 to 11 of α-MSH (ie α-MSH⁴⁻¹¹), wherein Gly¹⁰ has been deleted and substitutions incorporated at positions 4, 5, 6 and 7. The amino acid sequence of PG-901 and PG-911 are as follows:

PG-901
(SEQ ID NO: 1)
Ac-Nle-c[Asp-Pro-D-Nal(2′)-Arg-Trp-Lys]-NH₂
PG-911
(SEQ ID NO: 2)
Ac-Nle-c[Asp-Hyp-D-Nal(2′)-Arg-Trp-Lys]-NH₂

The methods of the invention may, in some embodiments, involve the administration of an MC5R-specific agonist selected from the group consisting of PG-901, PG-911 and derivatives thereof (especially derivatives including an amino acid substitution or other variation at position 6, particularly those which do not result in any significant alteration of the biological activity of the agonist). Other analogues of α-MSH⁴⁻¹¹ that are specific to MC5R are also suitable.

Further examples of MC5R selective agonists are described in International Patent Publication Nos WO 93/37097 and WO 2009/105824.

The agents described herein can be administered in any manner that results in the desired outcome of reducing blood glucose levels. Suitable methods of administration, dosages and formulations will be well known to the person skilled in the art or may be determined using standard methods.

A dosage regimen incorporating any of the agents will normally be determined after considering a variety of factors including type, species, age, weight, sex and physical condition of the subject; the route of administration; and/or the particular agent employed. An ordinarily skilled physician or veterinarian can readily determine an effective amount of the agent.

The agent may be administered in the form of a drug to a human or an animal. Alternatively, the agent may be incorporated into a food or beverage.

Suitable methods of administering the agent include, but are not limited to, intramuscular, intrathecal, intradermal, intraperitoneal (ip), intravenous (iv), subcutaneous (sc), intranasal, epidural, intradural, intracranial, intraventricular, and oral routes. Convenient routes for administration include, for example, infusion or bolus injection, topical, absorption through epithelial or mucocutaneous linings, ophthalmic, nasal, and transdermal. Administration can be systemic or local.

Suitable dosage forms include, without limitation, solid dosage forms and solid modified-release drug delivery systems (eg powders and granules, capsules, and/or tablets); semi-solid dosage forms and transdermal systems (eg ointments, creams, and/or gels); transdermal drug delivery systems; pharmaceutical inserts (eg suppositories and/or inserts); liquid dosage forms (eg solutions and disperse systems); and/or sterile dosage forms and delivery systems (eg parenterals, and/or biologies). Particular exemplary dosage forms include aerosols (including metered dose, powder, solution, and/or without propellants); beads; capsule (including conventional, controlled delivery, controlled release, enteric coated, and/or sustained release); caplet; concentrate; cream; crystals; disc (including sustained release); drops; elixir; emulsion; foam; gel (including jelly and/or controlled release); globules; granules; gum; implant; inhalation; injection; insert (including extended release); liposomal; liquid (including controlled release); lotion; lozenge; metered dose (eg pump); mist; mouthwash; nebulisation solution; ocular system; oil; ointment; ovules; powder (including packet, effervescent, powder for suspension, powder for suspension sustained release, and/or powder for solution); pellet; paste; solution (including long acting and/or reconstituted); strip; suppository (including sustained release); suspension (including lente, ultre lente, reconstituted); syrup (including sustained release); tablet (including chewable, sublingual, sustained release, controlled release, delayed action, delayed release, enteric coated, effervescent, film coated, rapid dissolving, slow release); transdermal system; tincture; and/or wafer. Typically, a dosage form is a formulation of an effective amount (such as a therapeutically effective amount) of the agent with pharmaceutically acceptable excipients and/or other components (such as one or more other active ingredients). The term “pharmaceutically acceptable” means approved by a regulatory agency of the federal or a state government or listed in the US Pharmacopoeia or other generally recognised pharmacopoeia for use in animals, and, more particularly, in humans. Excipients for use in exemplary formulations include, for example, one or more of the following: binders, fillers, disintegrants, lubricants, coatings, sweeteners, flavors, colorings, preservatives, diluents, adjuvants, and/or vehicles. In some instances, excipients collectively may constitute about 5%-95% of the total weight (and/or volume) of a particular dosage form.

Pharmaceutical excipients can be, for example, sterile liquids, such as water and/or oils, including those of petroleum, animal, vegetable, or synthetic origin, such as peanut oil, soybean oil, mineral oil, sesame oil, and the like. Water is an exemplary carrier when a formulation is administered intravenously. Saline solutions, blood plasma medium, aqueous dextrose, and glycerol solutions can also be employed as liquid carriers, particularly for injectable solutions. Oral formulations can include, without limitation, pharmaceutical grades of mannitol, lactose, starch, magnesium stearate, sodium saccharine, cellulose, magnesium carbonate, and the like. A more complete explanation of parenteral pharmaceutical excipients can be found in Remington, The Science and Practice of Pharmacy, 19th Edition, Philadelphia, Pa.: Lippincott Williams & Wilkins, 1995, Chapter 95. Excipients may also include, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like. Other examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. A formulation, if desired, can also contain minor amounts of wetting or emulsifying agents, or pH buffering agents.

In some embodiments of the present invention, the agent is administered locally to one or more skeletal muscles of a subject. This may be achieved by, for example, local or regional infusion or perfusion, topical application (for example, wound dressing), injection, catheter, suppository, implant, and the like.

For oral administration, oral dosages of the agent will generally range between about 0.001 mg per kg of body weight per day (mg/kg/day) to about 100 mg/kg/day, and such as about 0.01-10 mg/kg/day (unless specified otherwise, amounts of active ingredients are on the basis of a neutral molecule, which may be a free acid or free base).

For administration by injection (eg intravenously or subcutaneous injection), a subject would receive an injected amount that would deliver the agent in approximately the quantities described above.

A suitable composition may be intended for single daily administration, multiple daily administration, or controlled or sustained release, as needed to achieve the most effective results. However, notwithstanding the above, it will be understood by the person skilled in the art that the administered amount and frequency of administration for any particular subject may be varied and will depend upon a variety of factors including the activity of the particular agent, the metabolic stability and length of action of the agent, the age, body weight, general health, sex, diet, mode and time of administration, rate of excretion of the agent, and the severity of the disease or condition to be treated.

A gene therapy agent which increases the activity of and/or expression of AMPK in a subject may be a polynucleotide molecule comprising a polynucleotide sequence encoding AMPK. The term “polynucleotide molecule” and related terms including “polynucleic acid” and “nucleic acid” refers to deoxyribonucleic acid (“DNA”) and ribonucleic acid (“RNA”) in all their forms (ie single and double-stranded DNA, cDNA, mRNA, and the like).

Methods for transfecting cells, in particular muscle cells, with polynucleotide molecules are well known to the person skilled in the art. For example, constructs that include the polynucelotide molecule, an origin of replication, and a promoter can be used to introduce the polynucleotide molecule into cells for expression and/or replication. Selection and use of such constructs are well known to the person skilled in the art and will vary in accordance with the cell targeted to receive the polynucleotide molecule (see, for example, Sambrook, J et al., Molecular Cloning: A Laboratory Manual, Second Edition, Cold Spring Harbor (1989)). Exemplary constructs include plasmids, phage vectors, and the like. For example, a polynucleotide molecule which encodes AMPK may be inserted into E. coli plasmid vector PCRII™ (Invitrogen Corp, San Diego, Calif., United States of America) using suitable reagents.

Introduction of the construct into appropriate muscle cells enables expression of the cloned polynucleotide sequence. Appropriate expression vehicles are well known to the person skilled in the art and include those that are replicable in eukaryotic cells and/or prokaryotic cells and those that remain episomal or those which integrate into the host genome (see, for example, Sambrook, J et al., Molecular Cloning: A Laboratory Manual, Second Edition, Cold Spring Harbor (1989)). Suitable vehicles for expression of the polynucleotide sequences in eukaryotic host cells, particularly mammalian cells, include Rexp (with an RSV LTR, Moloney murine leukemia virus LTR driven expression vector), and the like.

The method of the first aspect may be suitable for treating a disease or condition associated with hyperglycaemia such as, for example, obesity (particularly diet induced obesity (DIO)), weight gain, Type II diabetes mellitus, insulin sensitivity, impaired glucose tolerance, and inflammation.

The method of the present invention also contemplates the administration of two or more agents selected from:

### (i) α-MSH;

(ii) an AMPK agonist;
(iii) an agent which increases the level of cAMP in skeletal muscle cells; and
(iv) an MC5R agonist.
The agents may be administered consecutively or concurrently. A particularly useful combination may comprise IBMX (an agent which increases the level of cAMP) and an MC5R agonist.

The method may further comprise administering to the subject one or more further agents for reducing blood glucose levels in, for example, a combination therapy. The further agents may be administered before, after or concurrently with the first mentioned agent. The further agents may be selected from, for example, agents well known to the person skilled in the art as being capable of reducing blood glucose levels such as insulin and insulin analogues including long-acting insulin analogues, sulphonylurea derivatives (a class of drugs used in the management of diabetes mellitus that act by increasing release of insulin from the beta cells of the pancreas) and metamorphins such as metformin (N,N-dimethylimidodicarbonimidic diamide) which improve hyperglycaemia through suppression of hepatic glucose production.

In a second aspect, the present invention provides a method of treating a disease or condition associated with hyperglycaemia in a subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or, increase the activity of and/or expression of AMPK in the muscle.

In a third aspect, the present invention provides a method of treating hyperglycaemia in an obese or overweight subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

In a fourth aspect, the present invention provides a pharmaceutical composition comprising an agent which: (i) increases the level of cyclic adenosine monophosphate (cAMP); and/or (ii) increases the activity of and/or expression of 5′ AMP-activated protein kinase (AMPK) in skeletal muscle of the subject optionally in combination with a pharmaceutically-acceptable carrier, diluent or excipient, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

In a further aspect, the present invention provides the use of an MC5R-specific agonist selected from the group consisting of α-MSH⁴⁻¹¹ analogues that are specific to MC5R such as PG-901, PG-911 and derivatives thereof (especially derivatives including an amino acid substitution or other variation at position 6, particularly those which do not result in any significant alteration of the biological activity of the agonist) for reducing blood glucose levels in a hyperglycaemic subject.

Similarly, the present invention provides the use of an MC5R-specific agonist selected from the group consisting of α-MSH⁴⁻¹¹ analogues that are specific to MC5R such as PG-901, PG-911 and derivatives thereof for treating a disease or condition associated with hyperglycaemia in a subject or for treating hyperglycaemia in an obese or overweight subject.

In a still further aspect, the present invention provides the use of an MC5R-specific agonist selected from the group consisting of α-MSH⁴⁻¹¹ analogues that are specific to MC5R such as PG-901, PG-911 and derivatives thereof (especially derivatives including an amino acid substitution or other variation at position 6, particularly those which do not result in any significant alteration of the biological activity of the agonist) in the manufacture of a medicament for reducing blood glucose levels in a hyperglycaemic subject.

Similarly, the present invention provides the use of an MC5R-specific agonist selected from the group consisting of α-MSH-⁴⁻¹¹ analogues that are specific to MC5R such as PG-901, PG-911 and derivatives thereof in the manufacture of a medicament for treating a disease or condition associated with hyperglycaemia in a subject or for treating hyperglycaemia in an obese or overweight subject.

In yet a further aspect, the present invention provides a method of identifying an agent capable of reducing blood glucose in a hyperglycaemic subject, wherein said method comprises the steps of;
(i) providing a cell or animal expressing melanocortin receptor type 5 (MC5R) or a composition or surface comprising MC5R;
(ii) contacting a test agent with said cell, composition or surface, or administering a test agent to said animal; and
(iii) detecting binding between said test agent and MC5R, or
(iv) in the case of said cell or animal, determining and comparing a response in said cell or animal with a control response.

The method may identify agents capable of providing a treatment of hyperglycaemia or a disease or condition associated with hyperglycaemia such as, for example, obesity (particularly diet induced obesity (DIO)), weight gain, Type II diabetes mellitus, insulin sensitivity, impaired glucose tolerance, and inflammation.

The control response referred to in step (iv) of the method may include a baseline response detected in said cell or animal without exposure to the test agent. The response to be determined and compared may be an increase in glucose uptake by said cell or animal, or an increase in the level of cyclic adenosine monophosphate (cAMP) and/or an increase in the activity of and/or expression of 5′ AMP-activated protein kinase (AMPK). In an animal, the response to be determined may be blood glucose levels 9ef following glucose challenge).

The test agent may be selected from known and novel compounds, complexes and other substances which may, for example, be sourced from private or publicly accessible agent libraries (eg the Queensland Compound Library (Griffith University, Nathan, QLD, Australia) and the Molecular Libraries Small Molecule Repository (NIH Molecular Libraries, Bethesda, MD, United States of America). The test agent may therefore comprise a protein, polypeptide or peptide (eg a α-MSH⁴⁻¹¹ analogue), or a mimetic thereof (including so-called peptoids and retro-inverso peptides), or a small organic molecule, especially those which comply or substantially comply with Lipinski's Rule of Five for “druglikeness”⁵. The test agent may also be selected on the basis of structural analysis of known or novel compounds or may otherwise be designed following structural analysis of MC5R binding sites.

The method may be adapted for high-throughput screening of large numbers of test agents. The step of comparing a response in said cell or animal with a control response may be conducted using one or more standard binding assay formats (eg ELISA-based or competition-based assays). Preferably, the test agent will be labelled with a readily detectable label (eg a fluorochrome or radioisotope) to allow detection of binding to, for example, a calcium channel receptor. A change in activity may be observed in such assays by using standard methods including spectrophotometric, fluorimetric, calorimetric or chemiluminescent means preferably providing for the automation or partial automation of the detecting step (eg by a microplate reader or use of a flow cytometer).

The invention is hereinafter described by way of the following non-limiting examples and accompanying figures.

### EXAMPLES

### Example 1

### Glucose and Insulin Treatments Stimulate α-MSH Release from the Pituitary Gland

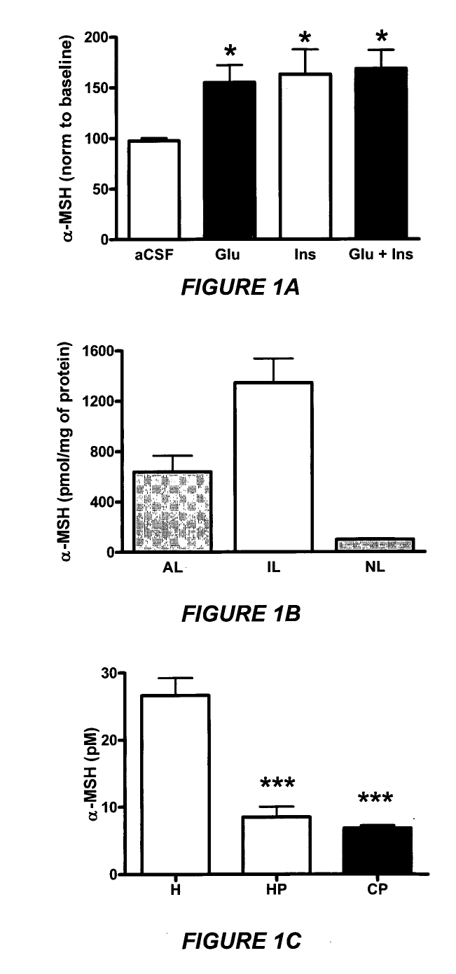

Adult monkey pituitaries (n=22) were halved and then treated separately with artificial cerebrospinal fluid (aCSF) equilibrated with 95% O₂ and 5% CO₂ and incubated at 37° C. After 1 hr equilibration period, halves were incubated for 45 min in aCSF (basal period) before being challenged with glucose (Glu): 15 mM, insulin (Ins): 200 nM or Glu+Ins (15 mM+200 nM) treatments for 45 min. The results are shown in FIG. 1A. Further, using immunohistochemistry, it was found that anti-α-MSH antibody (#AB5087; Millipore Biosciences Inc, Temecula, Calif., United States of America) bound to both the intermediate and anterior lobes (IL and AL) of monkey pituitaries (data not shown), indicating that the pituitary gland is an important contributor to circulating α-MSH. This was confirmed by measurement of α-MSH in monkey pituitaries by radioimmunoassay after acidic extraction (FIG. 1B).

In an additional experiment, α-MSH levels were assessed in children with hypopituitarism (HP) and after craniopharyngioma surgery (CP), relative to healthy children (H). 27 normal weight children served as controls (16 girls and 12 boys, X age=10 years). There were 15 patients with craniopharyngioma (ie a tumour in the hypothalamic-pituitary region, 9 girls, 6 boys, X age=13.8 years). The participating patients with CP were involved in a German pediatric study, KRANIOPHARYNGEOM 2000. All studied patients had undergone cranial tumour surgery in which all or most of the pituitary gland was removed. A complete tumour resection had been obtained in few patients, while 11 (73%) received percutaneous cranial irradiation. CP and pan-hypopituitarism patients were assessed for hormonal deficiencies and were adequately treated as required. The results are shown in FIG. 1C. It was observed that the HP and CP children had greatly reduced levels of circulating α-MSH. Considered together, the results of this example, indicate that the pituitary is the main source of plasmatic α-MSH.

### Example 2

### α-MSH Levels Increase in Response to Oral Glucose Administration in Both Healthy (Normal Body Weight) and Obese Humans

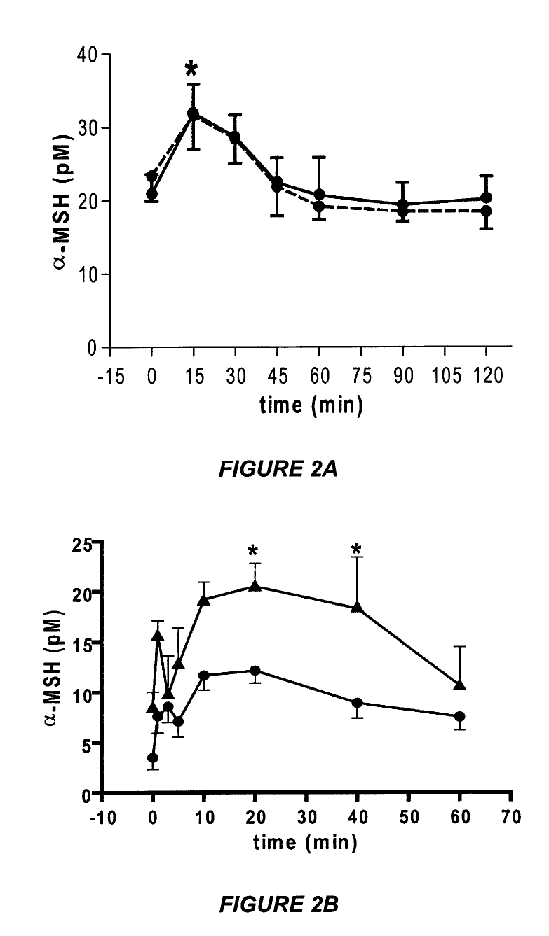

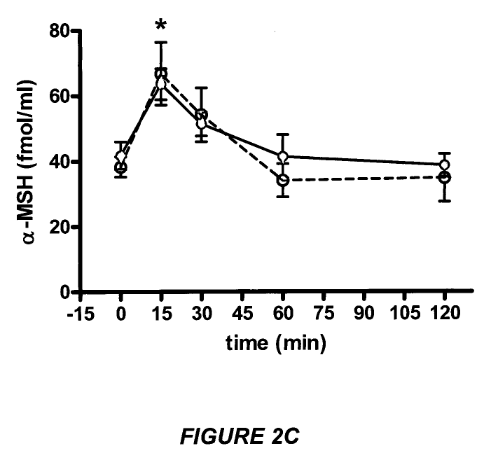

α-MSH levels were determined in 12 children treated at the Department of Pediatrics, University of Bonn (6 obese subjects: BMI>97th percentile versus 6 normal weight subjects, BMI 25-75th percentile) before and at different time-points after oral glucose loading (1.75 g/kg, max 75 g). *P<0.05 vs baseline value in the corresponding group by repeated-measures ANOVA. The results are shown in FIG. 2A.

### Example 3

### Obese Monkeys Have a Higher α-MSH Response to a Glucose Challenge than Controls

Female Japanese macaques (ages 5-7) were placed on either a control diet (13% of calories from fat) or High Fat Diet (HFD) (35% calories from fat plus calorically dense treats) and followed over 3 years. Body weight remained stable in the control animals throughout all 3 years. 5/9 animals showed a significant diet-induced obese phenotype compared to controls (11.6±2.4 kg vs. 8.7±0.2 kg) while the other 4 were resistant to diet-induced obesity. Intravenous Glucose Tolerance Tests (IVGTTs) were performed at the end of year 3 on monkeys fed regular chow and obese on HFD. Monkeys were tested after an overnight fast. Glucose (50% dextrose solution) was administered at a 0.6 g/kg dose by continuous infusion over 1 min via the small saphenous vein. α-MSH levels were measured during the intravenous glucose tolerance test in control (, n=6) and obese (▴, n=5) monkeys. *P<0.05 vs baseline value in the respective group by repeated-measures ANOVA. The AUC was significantly higher in obese than control monkeys. The results are shown in FIG. 2B.

### Example 4

### Control and Obese Mice (DIO) Showed a Similar Increase of α-MSH Levels in Response to Glucose Administration, to that Observed in Children

C57BL/6J mice were fed a regular diet (10% calories from fat) or a high fat diet (HFD, 45% calories from fat)) for 20 weeks. After a 14 h fast, intaperitoneal Glucose Tolerance Tests (ipGTTs) (1 mg/g) were performed on all mice. Glucose was administered intra-peritoneally (ipGTT). *P<0.05 vs baseline value in the respective group by repeated-measures ANOVA. The results are shown in FIG. 2C.

### Example 5

### α-MSH Levels Flattened in Response to Glucose Administration in POMC-Kir6.2 Mutant Mice but Not in Control Littermates

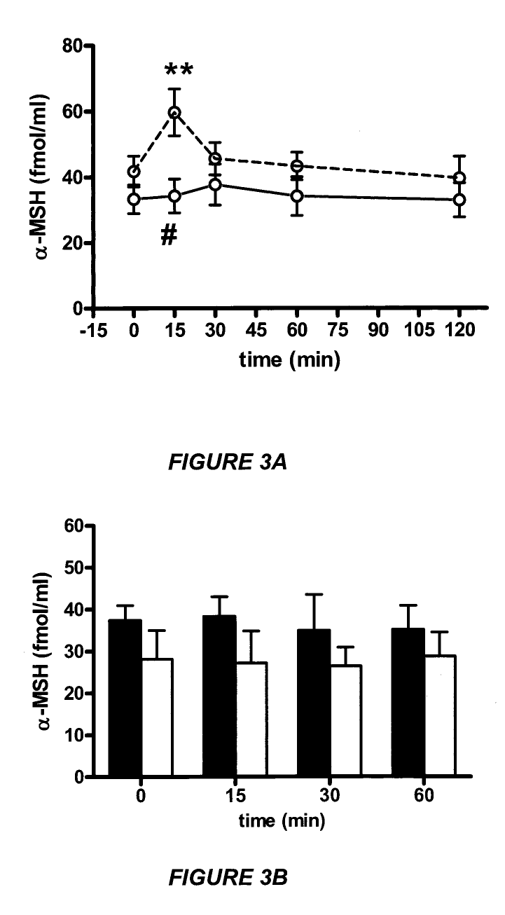

POMC-mutKIR6.2 mice is a mutant in which all POMC cells lack the capacity to sense glucose due to dysfunctional ATP/K+ channels. Mutants and wild-type littermates were fed a regular diet. Blood glucose and α-MSH samples were taken after ip injection of human insulin (1.0 U/kg). The results are shown in FIG. 3A. **P<0.01 vs baseline value in the respective group by repeated-measures ANOVA. # P<0.01 vs control littermate at the respective point by single measured. As shown in FIG. 3B, α-MSH levels do not change in response to insulin neither in POMC-mutKir6.2 mice (light bars) nor in control littermates (dark bars).

### Example 6

### Systemic α-MSH Infusion (External Jugular Vein) Increases Post-Prandial Muscle Temperature and Direct α-MSH Infusion into Femoral Artery also Causes an Increase in Muscle Temperature in Sheep

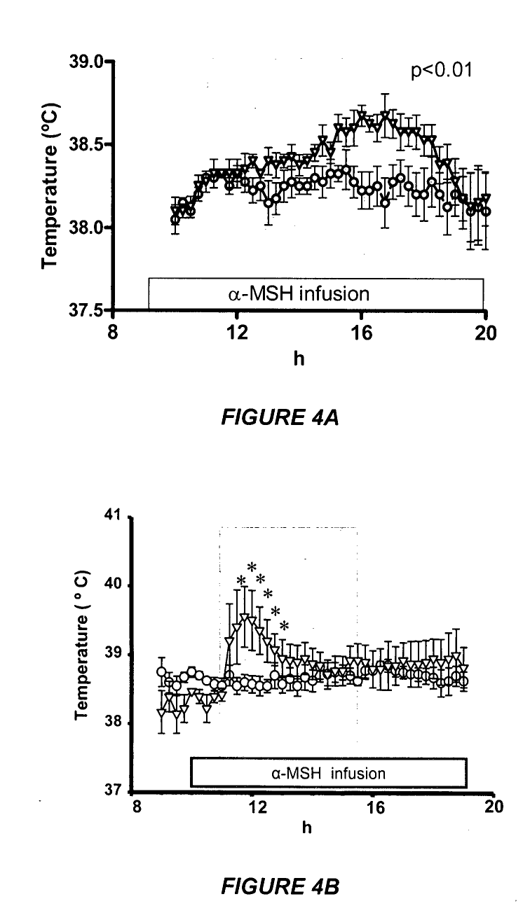

Four ovariectomised ewes were placed on a diet of temporal food restriction where food was made available between 11:00-16:00 h (as depicted by the grey box in FIGS. 4A and 4B). Animals were exposed to this feeding window for at least 2 weeks prior to experimentation. Then, one week before the onset of experiments, an indwelling cannulae was surgically inserted into the external jugular vein (experiment a) or femoral artery (experiment b). At the same time, a datalogger was implanted into the vastus lateralis muscle of the hindlimb and programmed to read temperature at 15 min intervals. Animals were treated with either α-MSH 100 μg/h (▾ triangles) for experiment a or 1 μg/h for experiment b or saline (800 μl/h, ). Infusions were carried out between 10:00-19:00 h. P<0.01 by AUC analysis. *P>0.05 compared to pre-treatment.

### Example 7

### Systemic α-MSH Infusion (External Jugular Vein) Increases Glucose Disposal During ipGTT in Control Mice but Not in Obese (DIO) Mice, and the Increases in Glucose Disposal Cannot be Prevented by Block of Central MC4R

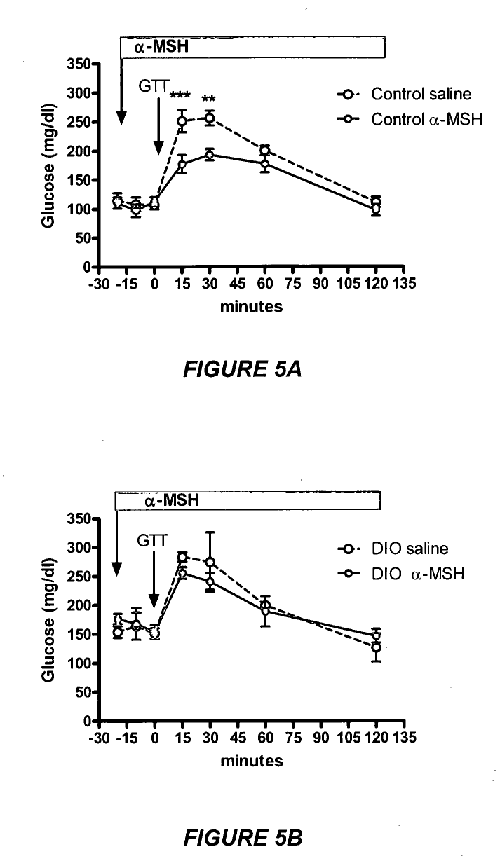

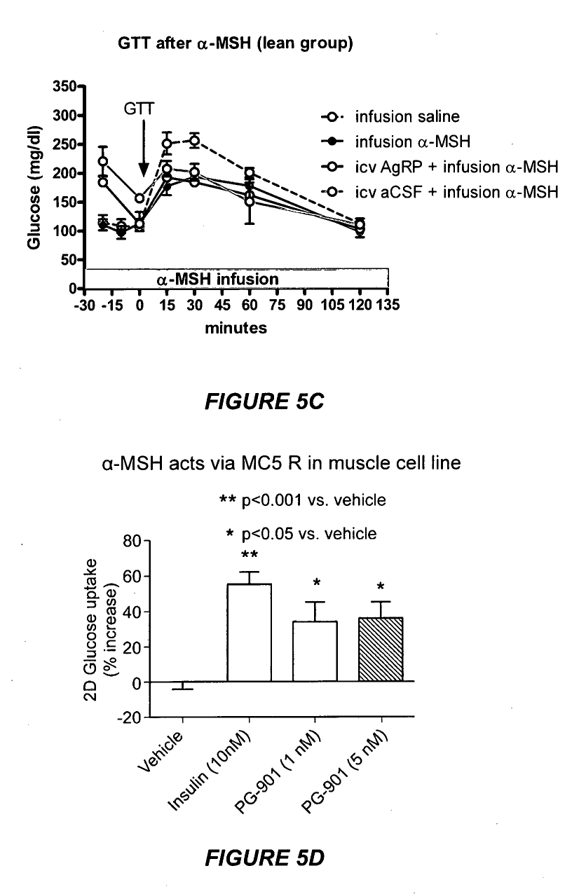

Mice were instrumented with an arterial catheter (carotid artery) to monitor blood pressure (BP), heart rate (HR) and for sampling. At the same time, a venous catheter (jugular vein) was placed to infuse α-MSH (1 μg/h) for 3 h. Blood glucose was measured before and at 10 and 20 mins of α-MSH infusion. After that, mice received an ip glucose injection (GTT, 1 mg/g). Blood glucose levels were measured at 15, 30, 60 and 120 mins after glucose challenge. BP and FIR were recorded during whole procedure. The results are shown in FIGS. 5A and 5B. **P>0.01. ***P>0.001 vs saline infusion. In a further experiment, MC4R activity was blocked using agouti-related peptide (AgRP; 1.0 nmol, 1 μl) administered (by injection to the lateral ventricle) 1 hr before commencement of the α-MSH infusion. The results are shown in FIG. 5C. The results clearly show that icy AgRP does not prevent the response to α-MSH infusion. Notably, it was also found that MC4R KO mice have an intact response to α-MSH infusion (data not shown). Also, in an experiment using the highly selective MC5R agonist denoted PG-901⁴ (1 nM and 5 nM amounts), it was found that increases of glucose uptake in skeletal muscle cells of about 30-40% (FIG. 5D) were unaffected by the presence of an MC3R and MC4R antagonist (data not shown), thereby providing further evidence that α-MSH acts via MC5R in muscle cells.

### Example 8

### α-MSH Increases Glucose Uptake in Soleus Muscles from Control Mice but Not from Obese Mice when Incubated in the Presence and Absence of Insulin

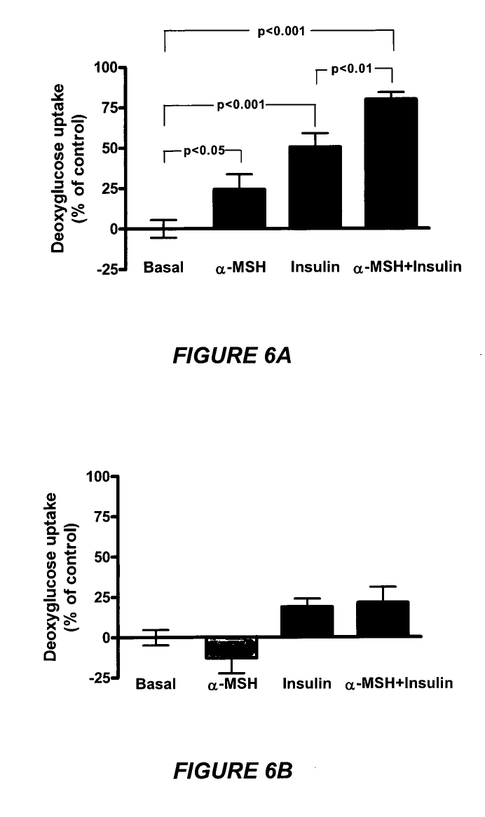

Mice were fasted overnight and soleus muscles were dissected from anaesthetised mice. Muscles were preincubated for 30 min with 1 ml of warmed (30° C.), pregassed (95% O₂-5% CO₂, pH 7.4), modified Krebs-Henseleit buffer supplemented with 2 mmol/l sodium pyruvate, 8 mmol/l mannitol, and 0.1% wt/vol BSA and were then incubated with or without 10 nM insulin, α-MSH (100 nM) or α-MSH plus insulin (100 nM+10 nM) for 20 min. Glucose uptake was assessed for 10 min using 2-deoxy-D-[2,6-³H]glucose (1 mmol/1, 0.5 μCi/ml) in the presence or absence of 10 nM insulin. Radioactivity was measured in muscle lysates by liquid scintillation counting. The results are shown in FIGS. 6A and 6B.

### Example 9

### MC5R mRNA Expression was Similar in Control and Obese Mice in Baseline Conditions

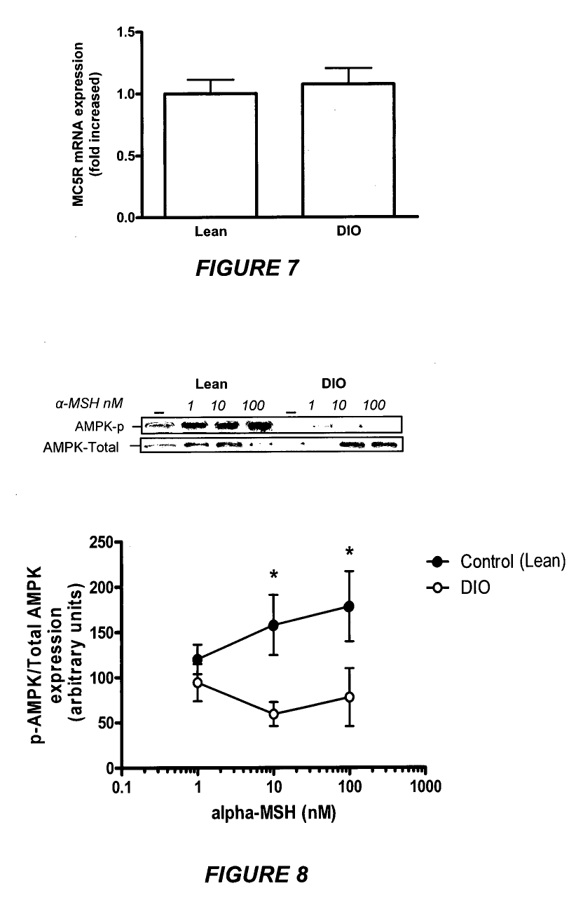

MC5R mRNA expression was assessed in soleus muscles of both control and DIO mice by RT-PCR. The results are shown in FIG. 7

### Example 10

### α-MSH Treatment in Vitro Causes a Dose-Dependent Increase of p-AMPK Muscle Levels from Lean but Not Obese Mice

Soleus muscles were dissected from anaesthetised mice. Muscles were incubated with α-MSH (at 1, 10, and 100 nM) for 45 min. After that, muscles were immediately homogenised in cold RIPA lysis buffer. Homogenates were centrifugated and supernatant were collected to measure protein contents (BSA assay). 50 μg of each sample was loaded on a 10% Tris-Glycine pre-cast gel. A liver protein sample was run as a positive control. The gel was then transferred onto an Immobilon PVDF membrane for 2 h and after blocking for 1 h with 5% milk in TBST, was incubated o/n with anti-p-AMPK antibody (1:1000; Cell Signaling Technology, Inc, Danvers, Mass., United States of America), anti-total AMPK (1:1000, Cell Signaling Technology, Inc), or anti-GAPDH antibody (1:20000). To avoid interferences with total AMPK antibody, membranes were stripped before incubating with this antibody. Goat anti-mouse IgG horseradish peroxidase (1:50000, Santa Cruz Biotechnology, Inc, Santa Cruz, Calif., United States of America) was used as secondary antibody. The results are shown in FIG. 8.

### Example 11

### α-MSH Infusion Causes an Increase of cAMP Levels in Muscles from Control Mice but Not from Obese Mice

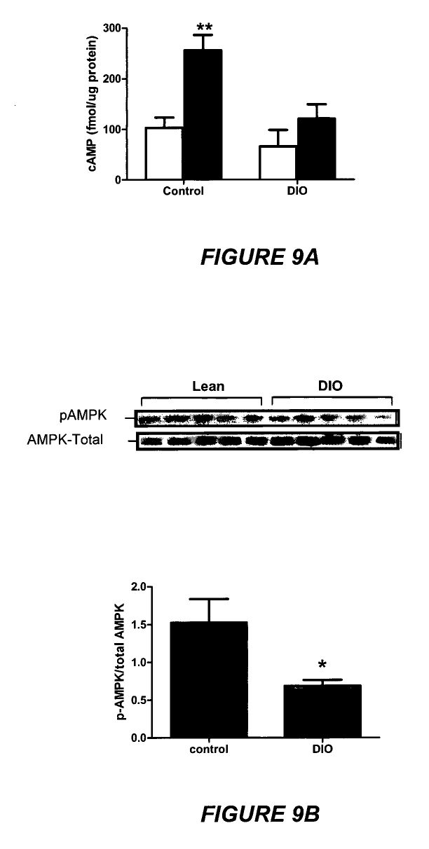

To measure cAMP concentrations and to determine p-AMPK protein expression in muscles, an experiment was conducted according to a similar design to that described in FIG. 5. Muscle tissues were obtained after 45 minutes of initiating α-MSH infusion and 25 minutes of administering glucose injection. cAMP concentration was determined with a direct immunoassay kit (BioVision Research Products, Mountain View, Calif., United States of America). The kit has a cAMP polyclonal antibody onto the plate. cAMP-HRP conjugates directly compete with cAMP from sample binding to the cAMP antibody on the plate. The HRP activity at 450 nm is inversely proportional to the cAMP concentration in samples. The results are shown in FIG. 9A. White bars: saline infusion, dark bars: α-MSH infusion

### Example 12

### p-AMPK Protein Expression is Increased in Muscles from Control but Not from Obese Mice after α-MSH Infusion

p-AMPK and total AMPK were determined by Western blot. Results shown in FIG. 9B are expressed as a ratio P/Total AMPK. *P<0.05

### Example 13

### Combination of α-MSH and Insulin Show Enhanced Increase in Glucose Uptake in Differentiated L6 Cells

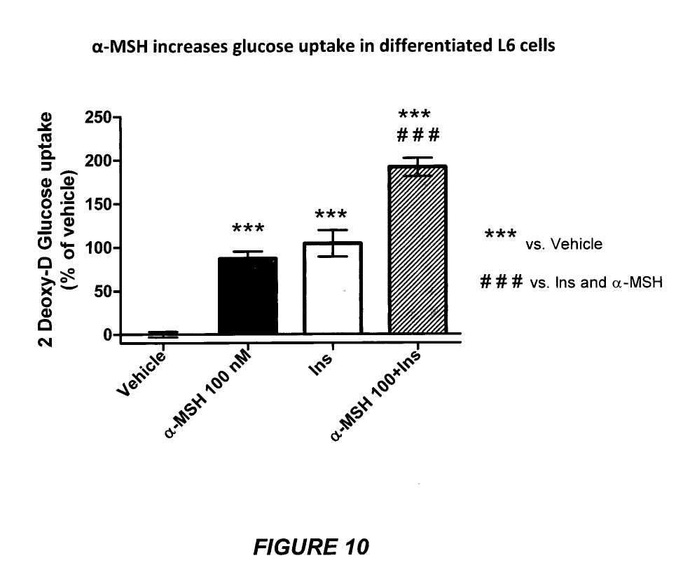

Rat L6 skeletal muscle cells were seeded in 12-well plates and differentiated in myotubes by replacing growth medium (DMEM+10% FBS) with DMEM+2% horse serum. Medium was changed every day for 1 week. Cells were preincubated for 20 min with no glucose medium+0.1% fatty acid-free BSA. 10 nM of insulin and/or 100 nM α-MSH was added to the corresponding wells. Glucose uptake was assessed for 15 min using 2-deoxy-D-[2,6-³H]glucose (1 mmol/1, 0.5 μCi/ml) in the presence or absence of 10 nM insulin and 100 nM of α-MSH. Radioactivity was measured in cell lysates by liquid scintillation counting. The results are shown in FIG. 10. It can be seen that the increases in glucose uptake achieved separately by α-MSH and insulin, are essentially complementary in the α-MSH plus insulin result.

### Example 14

### Prophetic Example—Effects of Various Agents on Blood Glucose Levels

The effects of agents including IBMX, AICAR, 8-Br-cAMP etc on blood glucose levels can be assessed as follows.

Mice can be instrumented with an arterial catheter (carotid artery) to monitor blood pressure, heart rate and for sampling. At the same time, a venous catheter (jugular vein) can be placed to infuse α-MSH, α-MSH+agent, and agent alone for a predetermined period (eg 3 h). Blood glucose can be measured before and at set times after infusion. After that, the mice receive an ip glucose injection (ipGTT, 1 mg/g). Blood glucose levels can then be measured at various times (eg 15, 30, 60 and 120 minutes) after glucose challenge.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Musi, N et al., Am J Physiol Endocrinol Metab 280:E677-E684 (2001).
2. Merrill, G F et al., Am J Physiol Endocrinol Metab 273:E1107-E1112 (1997).
3. Henin, N et al., Biochim Biophys Acta 1290:197-203 (1996).
4. Grieco, P et al., Biochem Biophys Res Commun 292(4):1075-1080 (2002).
5. Lipinski C A et al., Adv Drug Del Rev 46:3-26 (2001).

1. A method of reducing blood glucose levels in a hyperglycaemic subject, the method comprising administering an agent which: (i) increases the level of cyclic adenosine monophosphate (cAMP); and/or (ii) increases the activity of and/or expression of 5′ AMP-activated protein kinase (AMPK) in skeletal muscle of the subject, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

2. The method of claim 1, for treating a disease or condition associated with hyperglycaemia in a subject, said disease or condition being selected from the group consisting of: obesity, weight gain, Type II diabetes mellitus, insulin sensitivity, impaired glucose tolerance, and inflammation.

3.-4. (canceled)

5. The method of claim 1, wherein said method comprises administering a melanocortin-5 receptor (MC5R) agonist to one or more skeletal muscle cells of the subject.

6. The method of claim 5, wherein the MC5R agonist specifically activates MC5R.

7. The method of claim 5, wherein the MC5R agonist are selected from α-MSH⁴⁻¹¹ analogues.

8. The method of claim 7, wherein the MC5R agonist is PG-901 (Ac-Nle-c[Asp-Pro-D-Nal(2′)-Arg-Trp-Lys]-NH₂; SEQ ID NO: 1) or a derivative thereof.

9. The method of claim 1, wherein said method comprises administering an AMPK agonist which increases the activity and/or expression of AMPK in the skeletal muscle cells of the subject.

10. The method of claim 9, wherein the AMPK agonist is 5-aminoimidazole-4-carboxamide 1-β-D-ribonucleoside (AICAR).

11. The method of claim 1, wherein said method comprises administering an agent which increases the level of cyclic adenosine monophosphate (cAMP) in one or more skeletal muscle cells of the subject.

12. The method of claim 11, wherein the agent is a phosphodiesterase inhibitor.

13. The method of claim 12, wherein the phosphodiesterase inhibitor is 3-isobutyl-1-methylxanthine (IBMX).

14. The method of claim 11, wherein the agent is a protein kinase A (PKA) agonist.

15. The method of claim 14, wherein the PKA agonist is 8-bromoadenosine 3′,5′-cyclic monophosphate (8-Br-cAMP).

16. The method of claim 1, wherein said method comprises administering a MC5R agonist which specifically activates MC5R, and a phosphodiesterase inhibitor.

17. The method of claim 16, wherein the MC5R agonist is PG-901 or a derivative thereof, and the phosphodiesterase inhibitor is IBMX.

18. A pharmaceutical composition comprising an agent which: (i) increases the level of cyclic adenosine monophosphate (cAMP); and/or (ii) increases the activity of and/or expression of 5′ AMP-activated protein kinase (AMPK) in skeletal muscle of the subject optionally in combination with a pharmaceutically-acceptable carrier, diluent or excipient, wherein said agent is administered, adapted and/or formulated in a manner ensuring that an effective amount of said agent is delivered to the skeletal muscle cells so as to increase the level of cAMP and/or increase the activity of and/or expression of AMPK in the muscle.

19. A method of identifying an agent capable of reducing blood glucose in a hyperglycaemic subject, wherein said method comprises the steps of;
(i) providing a cell or animal expressing melanocortin receptor type 5 (MC5R) or a composition or surface comprising MC5R;
(ii) contacting a test agent with said cell, composition or surface, or administering a test agent to said animal; and
(iii) detecting binding between said test agent and MC5R, or
(iv) in the case of said cell or animal, determining and comparing a response in said cell or animal with a control response.

20. The method of claim 19, wherein the response to be determined and compared in step (iv) is an increase in glucose uptake by said cell or animal, or an increase in the level of cyclic adenosine monophosphate (cAMP) and/or an increase in the activity of and/or expression of 5′ AMP-activated protein kinase (AMPK).
